(19) United States
(12) Patent Application Publication
(10) Pub. No.: US 2024/0345095 A1
(43) Pub. Date: Oct. 17, 2024

(54) AN ASSAY FOR DETERMINING GASTROINTESTINAL PERMEABILITY BY DETERMINING A RATIO OF THE CONCENTRATION OF LACTULOSE TO THE CONCENTRATION OF GALACTOSE IN THE BLOOD SAMPLE

(71) Applicant: TriviumVet Designated Activity Company, Waterford (IE)

(72) Inventors: Louise GRUBB, Waterford (IE); Stuart FITZGERALD, Waterford (IE); Liam BYRNE, Waterford (IE); Serenia HORGAN, Waterford (IE)

(21) Appl. No.: 18/294,793

(22) PCT Filed: Jul. 21, 2022
(86) PCT No.: PCT/EP2022/070505

§ 371 Date: Feb. 2, 2024

(30) Foreign Application Priority Data
Aug. 3, 2021 (EP) 21189356.5

(51) Int. Cl. G01N 33/66; C12Q 1/00

(52) U.S. Cl. CPC G01N 33/66; C12Q 1/005; G01N 2800/062

(57) ABSTRACT

A method for detecting gastrointestinal permeability in a subject comprises administering a solution comprising lactulose and galactose to the subject. A sample of blood is taken from the subject after administering the solution. A ratio of the concentration of lactulose to the concentration of galactose in the blood sample is an indicator of gastrointestinal permeability.

### INTRODUCTION

The invention relates to an assay for determining gastrointestinal permeability. In one case the assay may be used for diagnosing gastric ulceration in equines, canines and other animals.

Gastric ulcer syndrome is an important cause of morbidity in equines, canines, and humans. 76% of elite event horses have gastric ulceration, between 98-100% of racehorses in training are affected and 50% of leisure horses also suffer from the problem. https://www.horseandhound.co.uk/horse-care/vet-advice/gastric-ulcers-dispelling-the-myths-282000#2epgJ1skq0AkahmY.99

Similarly, up to 75% of dogs receiving long-term nonsteroidal anti-inflammatory drug (NSAID) treatment have been reported to suffer from gastric ulceration. (https://onlinelibrary.wiley.com/doi/10.1111/jvim.16057)

Alterations in intestinal permeability in humans and animals are linked to many diseases including Crohn's disease, Protein-Losing Enteropathy (PLE) inflammatory bowel disease, celiac disease, irritable bowel syndrome, neoplasia, lymphangiectasia, non-alcoholic fatty liver disease, diabetes, Chronic Kidney Disease (CKD), Congestive Heart Failure (CHF) and in critically ill patients or patients undergoing major surgery. Moreover, increased intestinal permeability has recently been implicated in the pathogenesis of cardiovascular disease in several species. In addition to being a function of the disorders identified above; the development of increased intestinal permeability has been shown to be a result of complex environmental and lifestyle factors in other species. Increased permeability is implicated as both a causal and resultant feature of the aging process and the associated chronic systemic inflammatory state known as “inflammaging” in mammalian and lower species. Furthermore, the toxic effect of non-steroidal anti-inflammatory drugs (NSAIDs) on GI permeability is well documented in canine and human medicine, and this can progress to GI ulceration depending on the dose and duration of administration. Canine GI permeability is also known to be increased due to non-pharmaceutical or non-physiological mechanisms such as traumatic injury.

The only definitive diagnostic test available is endoscopy which requires general anaesthetic or sedation of the subject and visualisation of the gastric mucosa by an experienced veterinarian or doctor. The availability of a veterinarian or doctor to carry out the procedure, the risk of a general anaesthetic/sedation and the cost of the procedure make it prohibitive for use as a diagnostic tool in general practice. In consideration of these factors, it is common practice for veterinarians and doctors to prescribe treatments based on clinical presentation only. Assessment of complete ulcer resolution is challenging as chronic submucosal inflammation, disorganisation of stromal elements and transcriptomic derangements often persist in the setting of poor-quality ulcer healing, with a propensity for ulcer recurrence. These factors cannot be appreciated in clinical settings with current technologies.

Increased GI permeability in canine patients is often encountered in the context of ill-thrift or poor body condition score alongside apparently normal clinical pathology or chronic low-grade anaemia. If increased permeability or ulceration is suspected, endoscopy and/or biopsy is frequently required to pursue a diagnosis. Both procedures carry the additive risk of general anaesthesia, and serious surgical complications are not uncommon following biopsy. Some complications of gastroscopy and biopsy include perforation of the oesophagus, stomach, or duodenum; acute or delayed bleeding; pneumonia due to vomiting and aspiration; and rarely cardiac arrest or myocardial infarction.

Therefore, the development of an inexpensive screening test at the point-of-care would ensure early diagnosis and more accurate treatment of gastric ulceration and other conditions. Avoiding the need for sedation or general anaesthesia in diagnosing such conditions would eliminate risks such as adverse cardiac and neurological effects of sedative and anaesthetic drugs. Facilitating point-of-care confirmation of increased GI permeability would also aid in diagnosis and monitoring disease progression and treatment responses in both local and systemic disorders.

Urinary excretion ratios of orally administered lactulose and a monosaccharide over a 6-h period are currently employed in human medicine for measurement of small intestinal permeability. However, this approach does not allow characterisation of concentration-time curves that might aid in disease localisation, requires a high degree of patient compliance, and renders these tests impractical for widespread use in veterinary practice. Excretion ratios are highly dependent on renal function, which cannot be directly measured; estimated glomerular filtration rates based on blood levels of creatinine or other circulating substances are not precise, and renal tubular reabsorption of excreted solutes is not quantifiable and further confounds interpretation. Existing analytical methods also rely on use of a reference laboratory and cannot be applied in a point-of-care setting.

Lactulose-to-rhamnose urinary excretion ratios have previously been shown to distinguish healthy dogs from those with inflammatory bowel disease, small intestinal bacterial overgrowth, cancer, food intolerance, and dietary hypersensitivity, highlighting the broad spectrum of GI diseases in which assessment of permeability is clinically useful. However, measurement of urinary excretion levels is difficult and inconvenient, especially in non-human animals. As a non-nutrient monosaccharide, rhamnose is not actively absorbed and is reliant on transcellular diffusion for its absorption. Hence, rhamnose and similar non-nutrient sugars (such as mannitol) are incompletely absorbed, rendering them unsuitable for use as reference inputs in an oral absorption test. Furthermore, the assumption that passive absorption of non-nutrient sugars such as rhamnose and mannitol occurs mainly transcellularly cannot be upheld in cases of gastrointestinal pathology; rhamnose and mannitol also undergo increased paracellular absorption when this route is more available. Thus, lactulose excretion ratios are interpreted against an inconsistent denominator (rhamnose or mannitol) across disease states; as lactulose absorption and excretion increase with increased gastrointestinal permeability, so too do those of rhamnose and mannitol, reducing the utility of these ratios.

### SUMMARY OF THE DISCLOSURE

We describe various in vitro testing methods to determine a ratio of the concentration of lactulose to the concentration of galactose in a sample as an indicator of gastrointestinal permeability. A formulation containing lactulose and galactose is administered-preferably delivered orally/consumed by a subject. In one case the formulation is a solution containing both lactulose and galactose. A sample of blood is taken from the subject and tested in vitro to determine the ratio of the concentration of lactulose to the concentration of galactose in the blood sample.

We describe a method for detecting gastrointestinal permeability in a subject comprising the steps of:
administering a solution comprising lactulose and galactose to the subject;
taking a sample of blood from the subject after administering the solution; and
in vitro determining a ratio of the concentration of lactulose to the concentration of galactose in the blood sample as an indicator of gastrointestinal permeability.

Also described is a method for detecting gastrointestinal permeability in a subject comprising the steps of:
providing a sample of blood from a subject to which a solution comprising lactulose and galactose has previously been administered; and
in vitro determining a ratio of the concentration of lactulose to the concentration of galactose in the blood sample as an indicator of gastrointestinal permeability.

The solution containing lactulose and galactose is preferably delivered orally/consumed by the subject.

Because of its large molecular size (342 Da), lactulose does not readily permeate across healthy gastrointestinal mucosa, but it does cross the mucosa in the presence of gastrointestinal disease where the mucosa is damaged. If present in blood, lactulose is cleared via the urine; it is not metabolized, and the body does not produce it. Therefore, increased amounts of lactulose in blood after an oral dose is specific for increased gastrointestinal permeability. However, inferring permeability from point measures of lactulose is challenging due to variations in gastrointestinal transit time of oral solutions and potential variability in permeation rate at different anatomic sites.

Galactose is actively absorbed via a physiologic transport system and is therefore fully bioavailable, providing a known input against which lactulose absorption can be assessed. Following a period of fasting, negligible amounts of galactose are present in circulation. A similar mechanism of absorption applies to glucose and fructose; however, due to extremely high endogenous levels in blood, which mask oral inputs, these monosaccharides are unsuitable for use in this diagnostic test. Thus, galactose is the preferred monosaccharide-active absorption and low levels normally in circulation.

In health, galactose is transported across the intestinal enterocytes, while lactulose is not actively taken up. Galactose is therefore rapidly and completely absorbed in the small intestine. On the other hand, a low proportion of lactulose is absorbed into the systemic circulation. Interpretation of lactulose plasma concentration-time absorption curves in healthy subjects is hampered by variations in absorption between and within subjects. Once the concentration of both lactulose and galactose is determined, area under the curve (AUC) for lactulose and galactose are expressed as a ratio, AUC L:G (i.e. Lactulose:Galactose). This approach yielded a robust reference range in clinically normal subjects, corresponding to healthy intestinal absorption. We have found that AUC L:G provides optimum interpretability and consistency and is superior to other approaches including correlating rates of absorption, time to peak plasma concentrations, and absolute and relative peak concentrations of both lactulose and galactose.

Galactose is actively absorbed by the sodium-glucose linked transport system SGLT1, and absorption is active, complete, and overwhelmingly transcellular, rendering lactulose-to-galactose ratios more sensitive to the existence or progression of gastrointestinal pathologies.

Optionally, the method comprises the step of taking a plurality of blood samples over a period of time after administering the solution. Optionally, the period of time is at least 30 minutes or is at least 60 minutes. The method described is advantageous in comparison with quantification of lactulose and rhamnose or mannitol in urine as it determines gastrointestinal permeability within a much shorter period of time. We have found that early blood sampling following oral administration or consumption of lactulose and galactose is optimum. We have found that galactose absorption peaks within 15 minutes and remains present in plasma at measurable micromolar concentrations for approximately 60 minutes in the majority of individuals tested. Cumulative AUC of galactose therefore continues to rise throughout this period but does not change after 60 minutes. Lactulose absorption in health and disease states occurs over a longer period (up to 3 hours), but the rate of absorption slows after around 60 minutes.

After 60 minutes, the coefficient of variation (CV) for AUC L:G increases markedly in healthy dogs, and therefore the method becomes less useful as a screening or diagnostic aid. For example, in one group of healthy laboratory dogs, CV AUC L:G increased from 44% at 60 minutes to 102% at 120 minutes. Declining rate of absorption of lactulose coupled with renal clearance increasingly confounds interpretation of AUC L:G after 60 minutes, widening confidence intervals around any derived reference range, and necessitating greater deviations from the mean to allow inference of disease presence.

Optionally, the method comprises converting the blood sample into plasma and determining the concentration of lactulose and galactose in the blood plasma.

We have found that a determined ratio of the AUC of lactulose to the AUC of galactose of more than 1.1 in a blood plasma sample within 60 minutes of administration of the solution is indicative of increased gastrointestinal permeability. In this case, the ratio is calculated using AUC for both lactulose and galactose expressed as molar concentration multiplied by time (e.g. μM·h).

The ratio of AUC lactulose to AUC galactose can also be calculated using weight-per-volume multiplied by time units (e.g. mg·h/ml). In this latter case, the derived ratio will be approximately 1.9 times the value obtained using molar AUC ratios (1.9 being the ratio of the molecular weights of lactulose (342.296 g/mol) and galactose (180.156 g/mol)).

We have also found that a determined ratio of the AUC of lactulose to the AUC of galactose of more than 2.5 standard deviations from the reference mean in a blood plasma sample within 60 minutes of administration of the solution is indicative of increased gastrointestinal permeability.

Preferably, lactulose is administered in the solution at an amount of 0.2 to 1.0 gram per kg bodyweight of the subject. Preferably, lactulose is administered in the solution at an amount of 0.3 to 0.7 gram per kg bodyweight of the subject.

Preferably, galactose is administered in the solution at an amount of from 0.01 to 1.0 gram per kg bodyweight of the subject. Preferably, galactose is administered in the solution at an amount of from 0.05 to 0.5 gram per kg bodyweight of the subject.

Optionally, the solution comprises from 10% to 30% w/v of lactulose, or about 20% w/v of lactulose.

Optionally, the solution comprises from 1% to 5% w/v of galactose, and preferably the solution comprises from 1% to 3% w/v of galactose. Optionally, the solution comprises about 2% w/v of galactose. We have found that concentrations of galactose below 2% w/v in the solution are not optimal. As galactose is actively absorbed, it appears rapidly in plasma following oral administration. At concentrations below 2%, all galactose in the solution is rapidly depleted through absorption, yielding peak plasma concentrations within 5 minutes. Such rapid evolution of the plasma concentration-time curve mandates frequent early blood sampling in order to characterise a majority of this curve. Considering practicalities of solution consumption/administration and phlebotomy, the percent variation in target blood sampling times is much greater within this narrow 5-minute window. Such variation increases the likelihood that maximum plasma concentration will be mischaracterised, greatly impacting cumulative AUC measurements and rendering AUC L:G less valuable. Implementing repeated blood sampling around a 5-minute post-administration timepoint is undesirable and not practical in a clinical setting. At galactose concentrations above 2%, prolonged absorption and time to maximum plasma concentrations increase the length of time required to complete gastrointestinal permeability testing. At the optimal 2% w/v galactose concentration, peak plasma concentrations are reached between 10 and 15 minutes in the majority of subjects. Blood sampling at these timepoints allows both the test subject and care provider adequate time to prepare for phlebotomy and to alternate phlebotomy sites if necessary or desired.

Preferably the method comprises the step of waiting for a period of time after administering the solution before taking a blood sample, the waiting period after administering the solution and before taking a blood sample may be from 5 to 100 minutes, or 50 to 100 minutes. Optionally, the solution is administered after a fasting period, the fasting period may be from 6 to 24 hours, or 6 to 12 hours.

Optionally, the method comprises processing the lactulose and galactose in the blood sample to generate an output which is representative of the concentration of lactulose and galactose in the blood sample.

In one case, the method comprises converting lactulose in the blood sample into an entity including galactose and processing the galactose to generate an output which is representative of the concentration of lactulose in the blood sample. Optionally, the method comprises processing galactose in the blood sample to generate an output which is representative of the concentration of galactose in the blood sample.

Optionally, said processing comprises converting the galactose into D-galacto-hexodialdose and hydrogen peroxide in the presence of oxygen or a redox mediator and detecting one or more of the following which are provided:
quantity of hydrogen peroxide, and/or
quantity of D-galacto-hexodialdose, and/or
a change in the quantity of redox mediator, and/or
a change in the quantity of oxygen; and
processing the detected quantity or change in quantity to generate an output which is representative of the concentration of lactulose in the blood sample.

Optionally, the method comprises converting the galactose into D-galacto-hexodialdose and hydrogen peroxide and detecting one or more of the quantities of hydrogen peroxide, and/or the quantity of D-galacto-hexodialdose.

Optionally, the conversion of lactulose in the blood sample into galactose is an enzymatic conversion. Optionally, the enzyme for converting lactulose in the blood sample into galactose is β-galactosidase. Optionally, the conversion of the galactose into D-galacto-hexodialdose and hydrogen peroxide is an enzymatic conversion. Optionally, the enzyme for converting the galactose into D-galacto-hexodialdose and hydrogen peroxide is galactose oxidase. Optionally, the quantity of hydrogen peroxide is detected and processed to generate the output.

Preferably, an electrochemical biosensor system with a working electrode and a reference electrode is provided, at least one enzyme is immobilized on the working electrode surface and the conversion(s) are performed by contact with the immobilized enzyme or enzymes when the working electrode is exposed to the sample and a voltage is applied across the electrodes.

Optionally, the working electrode includes an enzyme mediator such as Pt or ruthenium purple and the enzyme(s) are drop cast onto the working electrode surface using a cross-linker, and preferably the cross-linker comprises glutaraldehyde, and/or silica sol-gel entrapment and/or co-disposition with polymer.

Preferably, for lactulose detection there are at least two immobilized enzymes including β-galactosidase and galactose oxidase. Optionally, hydrogen peroxide is oxidised on the working electrode surface. Optionally, the hydrogen peroxide is detected and said detection is by detection of the current flow in the sensor. Optionally, a change in the quantity of the redox mediator as a result of the conversion of the galactose into D-galacto-hexodialdose and hydrogen peroxide is detected and processed to generate the output.

Optionally, the system comprises at least two sensors linked to a common source of a sample such as via microfluidic channels, a first sensor having the immobilized enzymes and a second sensor having an immobilized enzyme galactose oxidase suitable for detection of galactose, and a processor is configured to omit galactose derived from lactulose.

Optionally, the system comprises only a single sensor, and said sensor has the immobilized enzymes, and said sensor is used at a different time for detection of galactose derived from lactulose.

Optionally, the redox mediator is one or more selected from ferrocene and its derivatives, quinone-based compounds, ferro/ferricyanide, or various redox organic polymers or inorganic complexes or combinations thereof, and the redox mediator may comprise osmium or ruthenium purple.

The sample may be a sample of blood plasma. The blood sample may be from a human. The blood sample may be from a non-human animal. The blood sample may be from an equine. The blood sample may be from a canine.

We also describe a method for determining a ratio of the concentration of lactulose to the concentration of galactose in a blood sample comprising the steps of:
a) converting lactulose in the blood sample into an entity including galactose;
b) converting the galactose provided by step (a) into D-galacto-hexodialdose and hydrogen peroxide in the presence of oxygen or a redox mediator;
c) detecting one or more of the following which are provided by step (b):
   quantity of hydrogen peroxide, and/or
   quantity of D-galacto-hexodialdose, and/or
   a change in the quantity of redox mediator, and/or
   a change in the quantity of oxygen;
d) converting galactose in the blood sample into D-galacto-hexodialdose and hydrogen peroxide in the presence of oxygen or a redox mediator;
e) detecting one or more of the following which are provided by step (d):
   quantity of hydrogen peroxide, and/or
   quantity of D-galacto-hexodialdose, and/or
   a change in the quantity of redox mediator, and/or
   a change in the quantity of oxygen;
f) processing the detected quantity or change in quantity provided by step (c) and step (e) to generate an output which is representative of the ratio of the concentration of lactulose in the blood sample to the concentration of galactose in the blood sample.

Optionally, the step (a) conversion of lactulose in the blood sample into galactose is an enzymatic conversion. Optionally, the enzyme for converting lactulose in the blood sample into galactose is β-galactosidase. Preferably, the steps (b) and (d) conversion of the galactose into D-galacto-hexodialdose and hydrogen peroxide is an enzymatic conversion. Optionally, the steps (b) and (d) enzyme for converting the galactose into D-galacto-hexodialdose and hydrogen peroxide is galactose oxidase. Optionally, the quantity of hydrogen peroxide detected in step (c) and (e) is processed in step (f) to generate the output.

Optionally, an electrochemical biosensor system with at least one working electrode and reference electrodes is provided, at least one enzyme is immobilized on each working electrode surface, the step (a) and (d) conversions are performed by contact with the immobilized enzymes when the working electrodes are exposed to the sample and a voltage is applied across the electrodes.

Optionally, the working electrodes each include Pt and the enzymes are drop cast onto the working electrode surface using a cross-linker and/or silica sol-gel entrapment and/or co-disposition with polymer. Optionally, the cross-linker comprises glutaraldehyde. Optionally, the immobilized enzymes include β-galactosidase and galactose oxidase for said step (a) conversion, and an immobilized enzyme galactose oxidase for said step (d) conversion.

Optionally, the hydrogen peroxide is oxidised on the working electrode surface in step (b).

Optionally, the hydrogen peroxide is detected in step (c), and said detection is by detection of current flow between the electrodes.

Optionally, a change in the quantity of the redox mediator as a result of the conversion of the galactose into D-galacto-hexodialdose and hydrogen peroxide is detected and processed to generate the output of step (c). Optionally, the redox mediator comprises one or more selected from ferrocene and its derivatives, quinone-based compounds, ferro/ferricyanide, or various redox organic polymers or inorganic complexes or combinations thereof, and the redox mediator may comprise osmium or ruthenium purple.

Preferably, the sample is a sample of blood plasma, which may be from a human or from a non-human animal. The blood sample may be from an equine or from a canine.

We also describe an apparatus for determining a ratio of the concentration of lactulose to the concentration of galactose in a blood sample, the apparatus comprising:
a first converter for:
   converting lactulose in the blood sample into an entity including galactose; and
   converting the galactose into D-galacto-hexodialdose and hydrogen peroxide in the presence of oxygen or a redox mediator;
a first detector for detecting:
   one or more of the following which are provided by the converter:
      quantity of hydrogen peroxide, and/or
      quantity of D-galacto-hexodialdose, and/or
      a change in the quantity of redox mediator, and/or
      a change in the quantity of oxygen;
a second converter for:
   converting galactose in the blood sample into D-galacto-hexodialdose and hydrogen peroxide in the presence of oxygen or a redox mediator;
a second detector for detecting:
   one or more of the following which are provided by the converter:
      quantity of hydrogen peroxide, and/or
      quantity of D-galacto-hexodialdose, and/or
      a change in the quantity of redox mediator, and/or
      a change in the quantity of oxygen; and
a processor for:
   processing the detected quantity or change in quantity provided
   by the detectors to generate an output which is representative of the ratio of the concentration of lactulose in the blood sample to the concentration of galactose in the blood sample.

Optionally, each converter comprises a substrate for an enzyme to perform conversion of lactulose in the blood sample into galactose. Optionally, the enzyme for converting lactulose in the blood sample into galactose is β-galactosidase. Optionally, each converter comprises a substrate for an enzyme for conversion of the galactose into D-galacto-hexodialdose and hydrogen peroxide. Optionally, the enzyme for converting the galactose into D-galacto-hexodialdose and hydrogen peroxide is galactose oxidase. Preferably, each detector is adapted to detect a quantity of hydrogen peroxide, and the processor is adapted to generate the output according to the detected quantity of hydrogen peroxide.

Optionally, the converter and the detectors are integrated in an electrochemical biosensor including for each detector a working electrode and a reference electrode, at least one enzyme is immobilized on each working electrode surface and each is adapted to perform the conversions by contact with the immobilized enzyme or enzymes when the working electrode is exposed to the sample, a driver to apply a voltage across the electrodes, and the processor is configured to process the electrical parameter signals to provide the output.

Optionally, each working electrode includes an enzyme mediator such as Pt and the enzymes are drop cast onto the working electrode surface using a cross-linker and/or silica sol-gel entrapment and/or co-disposition with polymer.

Optionally, the cross-linker comprises glutaraldehyde. Optionally, in the first converter there are at least two immobilized enzymes including β-galactosidase and galactose oxidase and in a second converter there is an immobilized enzyme galactose oxidase for galactose detection.

Optionally, the first and the second converters comprise a single working electrode with immobilized enzymes, and the single sensor is re-used with a time separation to act as first and second converters. Optionally, the driver is operable to cause hydrogen peroxide to be oxidised on each working electrode surface, and the detector is adapted to measure current flow arising from said applied drive.

Optionally, the processor is configured to subtract the concentration of galactose derived from lactulose from the concentration of galactose from the sample using a separate electrode to avoid double-counting.

Optionally, the processor is configured to compute areas under the curve (AUC) for lactulose and galactose concentrations. Optionally, the processor is configured to use any one or more of:
a linear trapezoidal Method in which linear interpolation between data points is used to calculate the AUC, in which an average concentration is applied to the entire time interval and the sum of the intervals results in the total exposure between the first and last time points, and dividing the total AUC by the total time elapsed to provide a concentration value,
a logarithmic trapezoidal method including logarithmic interpolation between data points to calculate the AUC, and
a linear-log trapezoidal method, being a combination of the linear and logarithmic trapezoidal methods.

We also describe a method for determining a ratio of the concentration of lactulose to the concentration of galactose in a blood sample comprising the steps of:
(a) converting galactose in the blood sample into D-galacto-hexodialdose and hydrogen peroxide in the presence of oxygen or a redox mediator;
(b) detecting one or more of the following which are provided by step (a):
   quantity of hydrogen peroxide, and/or
   quantity of D-galacto-hexodialdose, and/or
   a change in the quantity of redox mediator, and/or
   a change in the quantity of oxygen;
(c) converting lactulose in the blood sample into an entity including galactose;
(d) converting the galactose provided by step (c) into D-galacto-hexodialdose and hydrogen peroxide in the presence of oxygen or a redox mediator;
(e) detecting one or more of the following which are provided by step (d):
   quantity of hydrogen peroxide, and/or
   quantity of D-galacto-hexodialdose, and/or
   a change in the quantity of redox mediator, and/or
   a change in the quantity of oxygen; and
(f) processing the detected quantities or change in quantities provided by step (b) and step (e) to generate an output which is representative of the ratio of the concentration of lactulose in the blood sample to the concentration of galactose in the blood sample.

Optionally, steps (a) and (b) are carried out before steps (c) to (e).

Optionally, steps (a) and (b) are carried out at the same time as steps (c) to (e).

We also describe an apparatus for determining a ratio of the concentration of lactulose to the concentration of galactose in a blood sample, the apparatus comprising:
a converter for converting galactose in the blood sample into D-galacto-hexodialdose and hydrogen peroxide in the presence of oxygen or a redox mediator and for converting lactulose in the blood sample into an entity including galactose; and for converting the galactose from lactulose into D-galacto-hexodialdose and hydrogen peroxide in the presence of oxygen or a redox mediator;
a detector for detecting one or more of the following which are provided by the converter:
   quantity of hydrogen peroxide, and/or
   quantity of D-galacto-hexodialdose, and/or
   a change in the quantity of redox mediator, and/or
   a change in the quantity of oxygen; and
a processor for processing the detected quantity or change in quantity provided
by the detector to generate an output which is representative of the ratio of the concentration of lactulose in the blood sample to the concentration of galactose in the blood sample.

Preferably, the converter comprises
a first converter for:
   converting lactulose in the blood sample into an entity including galactose; and
   converting the galactose into D-galacto-hexodialdose and hydrogen peroxide in the presence of oxygen or a redox mediator; and
a second converter for:
   converting galactose in the blood sample into D-galacto-hexodialdose and hydrogen peroxide in the presence of oxygen or a redox mediator;
and wherein the detector comprises
   a first detector for detecting:
      one or more of the following which are provided by the first converter:
      quantity of hydrogen peroxide, and/or
      quantity of D-galacto-hexodialdose, and/or
      a change in the quantity of redox mediator, and/or
      a change in the quantity of oxygen; and
   a second detector for detecting:
      one or more of the following which are provided by the second converter:
         quantity of hydrogen peroxide, and/or
         quantity of D-galacto-hexodialdose, and/or
         a change in the quantity of redox mediator, and/or
         a change in the quantity of oxygen.

We also describe an apparatus for determining a ratio of the concentration of lactulose to the concentration of galactose in a blood sample comprising a first detector for detecting lactulose based on a probe such as a lectin that binds lactulose and a second detector for detection of galactose based on a probe such as a lectin that binds galactose, and a processor for quantifying the presence of lactulose and of galactose, and for generating an output which is representative of the ratio of the concentration of lactulose in the blood sample to the concentration of galactose in the blood sample. Methods for determining a ratio of the concentration of lactulose to the concentration of galactose in a blood sample using such an apparatus are also described.

In one example, each detector is an impedimetric detector configured to provide a response according to electrical impedance changes which arise upon binding of lactulose or galactose as applicable, or the extent of binding of complexes to binding sites which do not have bound lactulose or galactose after exposure to the sample.

In one example, each detector is an optical detector configured to provide a quantification response based on binding of lactulose or galactose, or the extent of binding of complexes to probes which do not have bound lactulose or galactose after exposure to the sample. This detector may comprise a competitive enzyme linked immunosorbent assay (ELISA),

In one case the method comprises detection of both galactose and lactulose based on 1) a lactulose-binding lectin and 2) a galactose-binding lectin. The quantitative detection of lactulose and galactose may be of any suitable mechanism to quantify the extent of galactose and lactulose which binds to a probe such as a lectin. The probe is preferably immobilized on a substrate. Preferably, the lectin is immobilized on a substrate in for example an assay well.

Quantification may be achieved by a competitive enzyme linked immunosorbent assay (ELISA), in which marker entities which are optically detectable may be provided after exposure of the probes to the sample, and these marker entities bind to any free lectin probes. The more of such bound marker entities which are present the smaller the extent of galactose or lactulose in the sample. For example, the marker entity may be a sugar and/or protein complex. When irradiated with radiation of a suitable wavelength range, preferably visible, the greater the optical response is the smaller the extent of the target galactose or lactulose is in the sample.

In another case the method comprises processing blood sample and testing by High Performance Liquid Chromatography (HPLC) followed by Mass Spectroscopy (MS) detection to generate an output which is representative of the concentration of lactulose and galactose in the blood sample.

Also described are various apparatus and methods as set out in the appended claims 1 to 88.

### BRIEF DESCRIPTION OF THE FIGURES

The invention will be more clearly understood from the following description thereof given by way of example only with reference to the accompanying Figures in which:

FIG. 1 is a dot plot of Lactulose:Galactose cumulative AUC in healthy dogs;

FIG. 2 is a plot of Lactulose absorption in healthy dogs over time;

FIG. 3 is a plot of Galactose absorption in healthy dogs over time;

FIGS. 4 and 5 are diagrams illustrating biosensors for use in in vitro measuring lactulose and galactose concentrations in a blood sample;

FIGS. 6 and 7 are plots of blood plasma spiked with 500 μM galactose or lactulose of a known concentration before use;

FIGS. 8(a) and 8(b) are photographs showing an area of ulcerated tissue in a dog (8a) and a similar area of healthy tissue (8b) in the same dog;

FIG. 9 is a diagram illustrating an impedimetric sensor system for use in in vitro measuring lactulose and galactose concentrations in a blood sample; and

FIGS. 10(a) to 10(d) are a series of diagrams illustrating steps in a method of capturing and quantifying galactose in a competitive enzyme linked immunosorbent assay (ELISA); and

FIGS. 11(a) to 11(d) are a series of diagrams illustrating steps in a method of capturing and quantifying lactulose in a competitive enzyme linked immunosorbent assay (ELISA).

### DETAILED DESCRIPTION

We describe a method for detecting gastrointestinal permeability in a subject comprising the steps of:
administering a solution comprising lactulose and galactose to the subject;
taking a sample of blood (for example plasma) from the subject after administering the solution; and
in vitro determining a ratio of the concentration of lactulose to the concentration of galactose in the blood sample as an indicator of gastrointestinal permeability.

A plurality of blood samples over a period of time after administering the solution may be used, the time being preferably at least 30 minutes.

Optionally, a determined ratio of the AUC of lactulose to the AUC of galactose of more than 1.1 in a blood plasma sample within 60 minutes of administration of the solution is indicative of increased gastrointestinal permeability.

Optionally, a determined ratio of the AUC of lactulose to the AUC of galactose of more than 2.5 standard deviations from the reference mean in a blood plasma sample within 60 minutes of administration of the solution is indicative of increased gastrointestinal permeability.

In one case a single biosensor system with a microfluidic channel may take a single sample and route it to the required biosensor for lactulose or galactose. The biosensor system will contain either one working electrode for the detection of lactulose and galactose or two working electrodes one for determining the proportion of lactulose and the other the proportion of galactose.

### Example 1—Healthy Laboratory Dogs: Proof of Concept

Quantification of plasma concentrations of lactulose (L) and galactose (G) has been performed in dogs using ion chromatography, over multiple timepoints up to 1 hour post administration (10, 20, 30, 60 minutes). Oral administration of solutions of up to 20% w/v lactulose and 2% w/v galactose concentrations and volumes of 2.5 ml/kg was well tolerated and did not result in appreciable effects on gastrointestinal function or faecal characteristics.

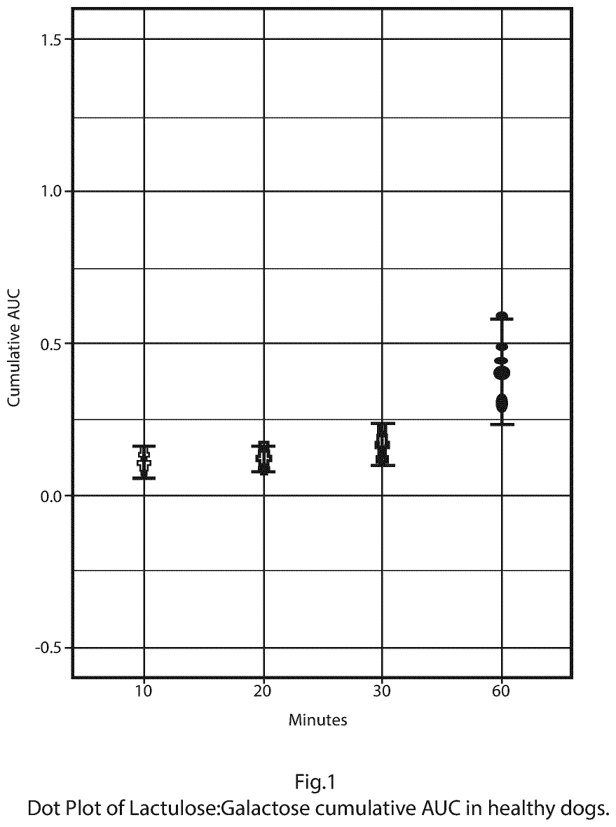

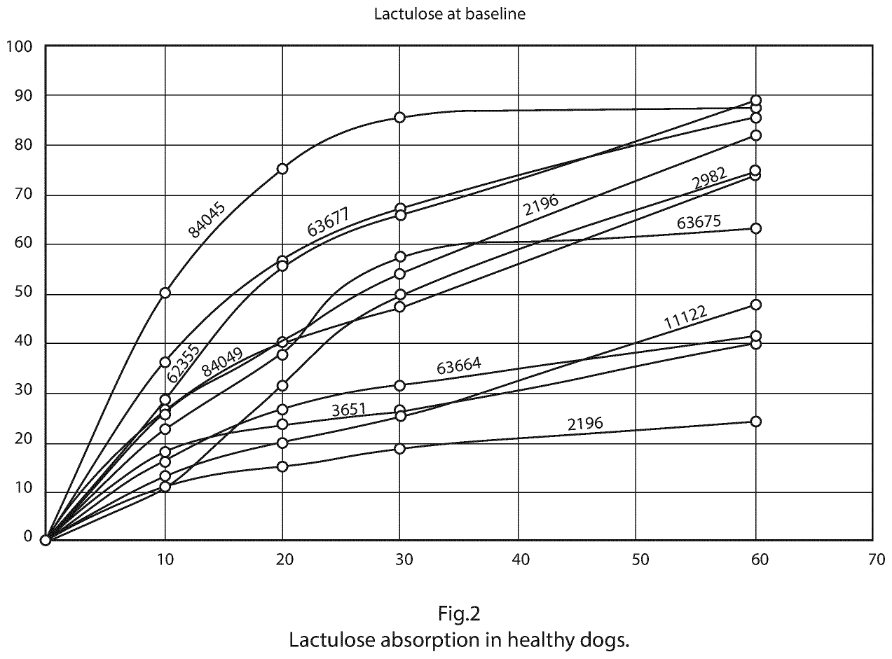

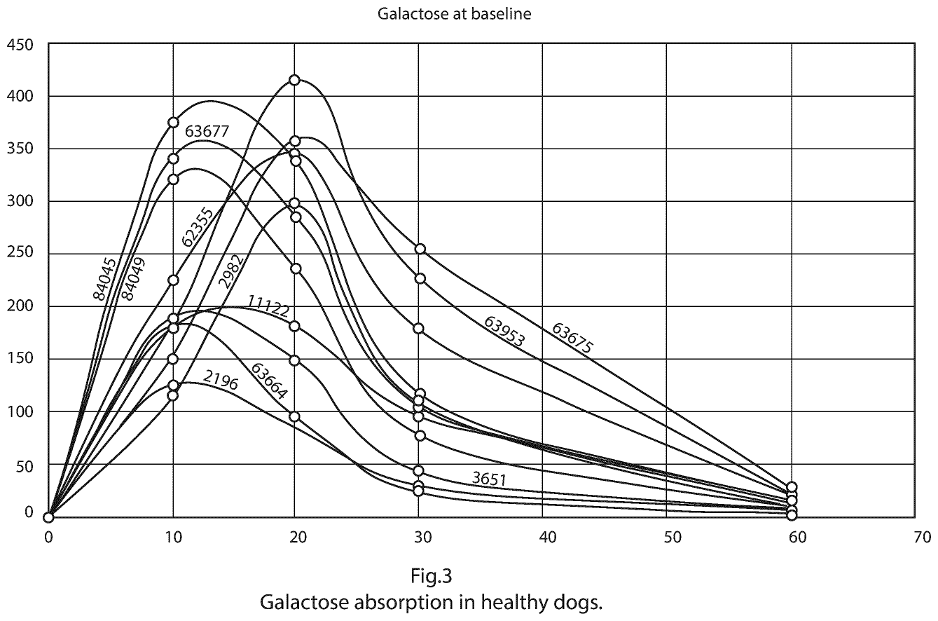

In health, dogs actively transport galactose across the intestine, while lactulose is not actively taken up. The galactose present in the lactulose and galactose solution is therefore rapidly and completely absorbed in the small intestine, with plasma concentration-time absorption curves yielding information on gastric exit times in individual animals (FIG. 3). On the other hand, a low proportion of lactulose is absorbed into the systemic circulation. Interpretation of lactulose plasma concentration-time absorption curves in healthy dogs (FIG. 2) is hampered by interindividual variability and variations between and within dogs in gastrointestinal transit times, though total absorption rates can be correlated with endoscopic findings. Once the concentration of both sugars is determined, area under the curve (AUC) measurements are made for lactulose and galactose in units of μM·h and these are expressed as a ratio, AUC L:G. This approach yielded a tight range of values in a group of 11 clinically normal dogs, corresponding to healthy intestinal absorption (FIG. 1 & Table 1). Alternative approaches to interpretation, including correlating rates of absorption, time to peak plasma concentrations, and absolute and relative peak concentrations of both sugars did not improve the interpretability or consistency of the results obtained. Further experimentation in laboratory dogs demonstrated that in health, AUC L:G 60 minutes after administration of a 20% lactulose/2% galactose solution did not exceed a 95% confidence level cut-off value of 1.1.

All values for AUC L:G 60 minutes following oral administration of the test solution lay within 2.5 standard deviations of the population mean Further studies with larger numbers of dogs will validate these cut-off values.

FIG. 1 is a dot plot of Lactulose:Galactose cumulative AUC in healthy dogs.

FIG. 2 is a plot of Lactulose absorption in healthy dogs over time.

FIG. 3 is a plot of Galactose absorption in healthy dogs over time.

TABLE 1

| Normal | | | 90% Lower | 90% Upper |
|---|---|---|---|---|
| reference | | | Reference | Reference |
| intervals | Lower | Upper | Limit | Limit |
| Measure- | Reference | Reference | Confidence | Confidence |
| Minutes | Limit | Limit | Interval | Interval |
| L:G-10 | 0.0571 | 0.1577 | 0.0343- | 0.1349- |
| | | | 0.0798 | 0.1805 |
| L:G-20 | 0.0803 | 0.1618 | 0.0618- | 0.1434- |
| | | | 0.0987 | 0.1803 |
| L:G-30 | 0.0989 | 0.2322 | 0.0688- | 0.2020- |
| | | | 0.1291 | 0.2624 |
| L:G-60 | 0.2340 | 0.5762 | 0.1566- | 0.4897- |
| | | | 0.3114 | 0.6536 |

Values significantly above those in the “upper reference limit” column are consistent with gastric ulceration and increased gastrointestinal permeability.

### Example 2—Detection of Ulceration

Subsequent to the generation of baseline values as outlined in Example 1, ulceration of the gastrointestinal tract was induced in the same laboratory dogs using supratherapeutic doses of a combination of NSAIDs. Plasma samples from the following procedures were processed using the methods described above at the same analytical laboratory at the same time as the baseline samples to minimize analytical variability.

Healthy laboratory dogs (14.0 to 16.7 kg) were screened to exclude individuals with clinically silent disorders, including gastrointestinal disease. Under a Good Clinical Practice (GCP) study protocol, each dog was administered 50 ml of a 20% w/v lactulose+2% w/v galactose solution orally (0.6-0.7 g/kg lactulose and ˜0.05 g/kg galactose Per Os (PO)) following a 24-h fast at baseline and following a period of ulcer induction. On each occasion, peripheral venous blood was drawn from each dog into 1.3-ml Sarstedt blood tubes containing lithium heparin as an anticoagulant at 10, 20, 30 and 60 minutes following bolus administration of the lactulose solution. The Sarstedt tube product number was 41.1393.005. Blood samples were centrifuged for 15 minutes at 3000 rpm, before decanting plasma into plain tubes; plasma was stored at −80° C. until analysis. Prior to administration of the lactulose/galactose solution, endoscopy was performed on each dog, and the presence and extent of gastric ulceration were assessed by a blinded specialist veterinary gastroenterologist. Following confirmation of the presence of ulceration, NSAID administration was discontinued, and post ulcer induction lactulose/galactose administration and plasma collection were performed.

The use of an anticoagulant was necessitated by the tendency of serum to congeal following lactulose administration. The presence of micromolar concentrations of lactulose in blood, consistent with levels expected to be achieved in canine and other patients with gastric ulceration were found to result in the formation of a serum gel when blood samples were collected into plain serum tubes. The viscosity of the serum gel so formed prevented dilution and analysis of these blood samples. The use of lithium heparin anticoagulant-coated tubes prior to centrifugation of blood samples yielded liquid plasma suitable for analysis. Other anticoagulants such as sodium citrate, sodium fluoride or potassium oxalate may also have been used for this purpose.

Blood samples were taken at 10, 20, 30 and 60 minutes post administration and the concentrations of Lactulose and Galactose were measured in the blood samples.

The ratios of AUC L:G in units of μM·h at 10, 20, 30 and 60 minutes post administration were calculated and are summarised below. Following ulcer induction, AUC L:G values are markedly increased above the baseline range previously established, reflecting pathologic increases in gastrointestinal permeability and aiding in clinical decision making and provision of appropriate specific (e.g. proton pump inhibition) and supportive (e.g. antimicrobial and dietary) therapies.

TABLE 2

| | Area under the curve Lactulose:Galactose (Mean (SD)) | | | |
|---|---|---|---|---|
| | 10 min | 20 min | 30 min | 60 min |
| Baseline | 0.11 (0.03) | 0.12 (0.02) | 0.17 (0.03) | 0.41 (0.09) |
| Post-ulcer | 0.78 (0.98) | 0.55 (0.45) | 0.60 (0.41) | 1.31 (0.83) |
| induction | | | | |
| Reference | 0.06-0.16 | 0.08-0.16 | 0.10-0.23 | 0.23-0.58 |
| range (see | | | | |
| Table 1) | | | | |

Integration of this absorption test with circulating albumin, globulin and total protein measurements was predictive of maximum weight loss following NSAID toxicity in this cohort of dogs. Incorporation of the absorption test ratio with a wider panel of clinical pathology parameters or other diagnostic modalities in cohorts of patients with defined disease states (syndromes) will facilitate the evaluation of whether intestinal absorption testing can improve diagnosis of occult intestinal disorders.

Based on the established baseline values, increases in AUC L:G correctly identified all dogs with ulceration as having increased intestinal permeability through elevated absorption ratios from ten to sixty minutes following administration of lactulose/galactose test solution. Moreover, the test was also shown to be sensitive to a less severe NSAID challenge and the subsequent effects on intestinal permeability.

In this case, the ratio is calculated using AUC for both lactulose and galactose expressed as molar concentration multiplied by time (e.g. μM·h). AUC lactulose to AUC galactose can also be calculated using weight-per-volume multiplied by time units (e.g. mg·h/ml). In this latter case, the derived ratio will be approximately 1.9 times the value obtained using molar AUC ratios (1.9 being the ratio of the molecular weights of lactulose (342.296 g/mol) and galactose (180.156 g/mol)). By applying a conversion factor of 1.9 (equivalent to the ratio of molecular weights of lactulose and galactose) the range of AUC L:G values obtained in the study could be readily transformed to allow interpretation of weight-per-volume-based AUC figures.

### Example 3—Optimisation of Oral Solution Composition and Blood Sampling Schedule

Under a GCP study protocol, seven apparently healthy laboratory dogs (four male and three female) weighing 10.95 to 15.85 kg were randomised to receive 2.5 ml/kg of either (a) 10% w/v lactulose+1% w/v galactose or (b) 20% w/v lactulose+2% w/v galactose solution on Study Day 0. Each dog then received the same dose of the alternate solution on Study Day 3. Blood samples were drawn 5, 10, 15, 30, 45, 60, 120, 180 and 240 minutes following dosing. The purpose of the study was to investigate the effect of varying lactulose/galactose concentration and osmotic gradient on absorption profiles, and to allow optimisation/minimisation of blood sampling to facilitate clinical use of the test.

TABLE 3

| | Study Day 0 | Study Day 3 |
|---|---|---|
| Group | Treatment 1 | Treatment 2 |
| 1 | 2.5 ml/kg bodyweight 10% | 2.5 ml/kg bodyweight 20% |
| | w/v lactulose/galactose | w/v lactulose/galactose |
| | solution administered orally | solution administered orally |
| Group | Treatment 1 | Treatment 2 |
| 2 | 2.5 ml/kg bodyweight 20% | 2.5 ml/kg bodyweight 10% |
| | w/v lactulose/galactose | w/v lactulose/galactose |
| | solution administered orally | solution administered orally |

Both treatments were well tolerated by all dogs, with no evidence of gastrointestinal distress. Whereas lactulose absorption as reflected in plasma levels increased steadily in most dogs after receiving the 20% w/v solution, absorption patterns were more irregular following the 10% solution. This difference may primarily reflect the difference in osmolarity between the two solutions, with the lower osmolarity less effectively driving absorption of the large lactulose molecule. Furthermore, galactose permeation/absorption curves displayed very early maximum concentrations (Cmax occurring at 5 mins in 6 dogs), and rapid elimination. Such rapidly evolving profiles increase the likelihood that AUC calculations will inaccurately reflect true absorption levels and decrease the value of the permeability test. From a practical perspective, sampling only 5 minutes following administration of the test solution is not optimal, as marked variation in speed of consumption may be expected under field conditions, and the proportional deviation from target sampling time is also likely to be large. These factors are reflected in the wide variance presented in Table 4 below.

Following administration of the 20% w/v lactulose+2% w/v galactose solution, Cmax of galactose was observed after 10 minutes for 4 dogs, 15 minutes for 2 dogs, and 45 minutes for 1 dog. Absorption curves were of a more consistent shape than for the 10% w/v solution, suggesting that they were more reflective of true absorption patterns. When pursuing the second aim of the study; i.e. optimisation of sampling schedules, it was found that administration of a 20% w/v lactulose+2% w/v galactose solution, followed by blood draws at 10, 15, 30 and 60 minutes allowed a high degree of accuracy in AUC L:G at 60 minutes, yielding a mean square error (MSE) of 0.007 when compared with the reference value derived from the full sampling schedule. This compared with a MSE of 17.108 for the same “optimised” sampling schedule following a 10% w/v administration. Blood draws at 10, 15, 45, and 60 minutes would yield MSE of 0.125 and 18.422 for the 20% and 10% solutions, respectively. AUC L:G is in units of μM·h.

Thus, it was determined that an optimised testing regimen for use in future clinical trials would comprise administering 2.5 ml/kg of a 20% w/v lactulose+2% w/v galactose solution orally, followed by blood sampling at 10, 15, 30 and 60 minutes later.

TABLE 4

| | 10% lactulose/ | 20% lactulose/ |
|---|---|---|
| | 1% galactose | 2% galactose |
| All data points | | |
| Mean (SD) AUC L:G at 60 mins | 2.47 (2.60) | 0.67 (0.28) |
| Coefficient of variation | 105% | 42% |
| 10, 15, 30, and 60 minute blood | | |
| draws only | | |
| Mean (SD) AUC L:G at 60 mins | 3.02 (2.80) | 0.63 (0.24) |
| Coefficient of variation | 93% | 38% |

The health of all 7 dogs was further investigated by administering video capsule endoscopes, which were later retrieved from the animals' faeces and the footage reviewed by a veterinary gastroenterologist. It was unfortunately not possible to retrieve a capsule from a single dog: despite repeating the procedure, this animal twice destroyed the capsule before the laboratory staff were able to retrieve it. No clinically significant lesions were found in the 6 video exams reviewed.

### Example 4—Detection of Clinically Silent Intestinal Lesions

A bolus of 2.5 ml/kg of a 20% lactulose/2% galactose solution was administered to an apparently healthy laboratory dog weighing 14.15 kg (total dose: 35.4 ml). Blood samples were drawn into 1.3-ml Sarstedt blood tubes containing lithium heparin 10, 15, 30, and 60 minutes following administration, and processed in the same analytical laboratory at the same time as those samples discussed under Example 3. AUC L:G is in units of μM·h. The 60-minute AUC L:G ratio for this dog, calculated as previously described, was 1.40—approximately 3.2 standard deviations from the mean value of 0.63 obtained from the previously discussed 7 dogs.

The health of the dog was further investigated by administering video capsule endoscopes, which were later retrieved from the animal's faeces and the footage reviewed by a veterinary gastroenterologist. The presence of multifocal ulcerative cauliflower-like lesions, clearly visible in the gastrointestinal tract of this dog, was not suspected based on clinical examination and traditional laboratory assessments; however, the significantly elevated AUC L:G ratio was indicative of this clinically silent disease state.

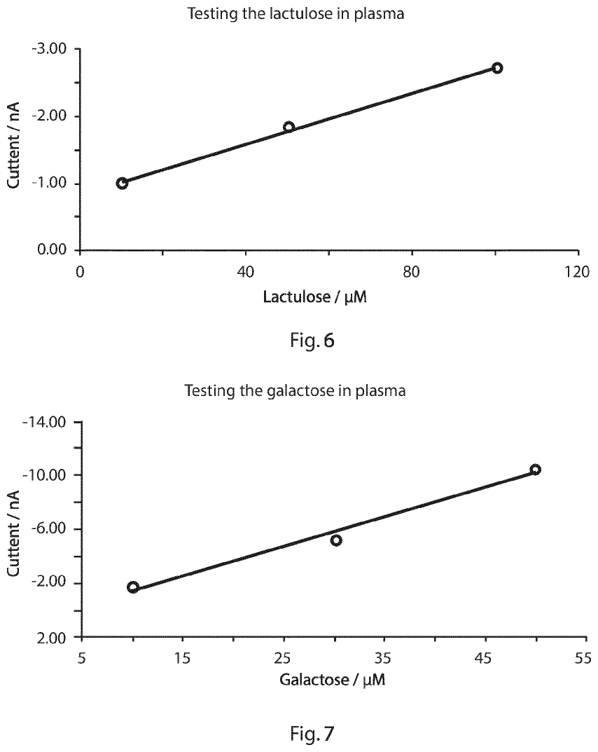

FIGS. 8(a) and 8(b) are photographs showing an area of ulcerated tissue (FIG. 8(a)) in the dog and a similar area of healthy tissue (FIG. 8(b)) in the same dog. In FIG. 8(a) the left-hand side 40% of the image shows ulcerated cauliflower-like appearance.

In the absence of clinical signs such as vomiting, diarrhoea, weight loss, gastrointestinal haemorrhage or abdominal pain, these lesions may reflect primary ulceration, benign mucosal polyps, localised parasitism, inflammation, or cyst formation. The dog remained asymptomatic in the period immediately following this investigation, and the laboratory staff responsible for its care elected not to pursue further investigation. However, had the staff elected to do so, it would have been possible to direct a traditional fibreoptic endoscope to the site of the lesions to allow biopsy and precise characterisation of the disease process. This would then allow an optimal treatment plan to be devised, whether medical or surgical in nature.

Presence of these abnormal intestinal lesions was not detectable by routine or non-invasive means, other than the animal's elevated AUC L:G ratio, demonstrating the sensitivity, utility and novelty of the described invention in companion animal or medical practice.

### Discussion

Quantifying the absorption of two sugars in this intestinal permeability test allows for the assessment of both passive and active transport across the intestinal surface. Moreover, relative changes in galactose and lactulose exposure following oral administration may indicate the potential for impaired active nutrient uptake (galactose) versus paracellular loss (lactulose) in various disease states. Continuous monitoring of weight changes in dogs with NSAID toxicity was performed and the final weight change (%) and maximal % weight change during the study period were calculated. The AUC L:G ratio values obtained by lactulose/galactose absorption testing at Day 6 were predictive of both these body weight outcomes at Day 28. Correlation values for the predictive capacity of Day 6 AUC L:G, with respect to final weight change and maximal weight loss were improved by incorporation into a regression model with total protein, albumin, and globulin; R2 values for final weight change (%) and maximal weight loss (%) were 0.90 and 0.95, respectively. Thus, intestinal absorption testing can be used to predict future weight changes during a period of disease and/or therapeutic intervention; potentially serving as a prognostic tool.

Intestinal absorption testing has been previously used to aid in the identification and diagnosis of adverse reactions/responses to NSAID use, small-intestinal bacterial overgrowth, CKD, occult intestinal bleeding, a variety of cancers, and PLE.

Evaluation of different oral solution concentrations and blood sampling schedules has allowed optimisation of both, and a 20% w/v lactulose+2% w/v galactose solution has demonstrated benefits over lower-concentration alternatives. Calculation of AUC L:G ratios over 60 minutes following administration of such a solution has allowed identification of an animal with naturally occurring, clinically silent intestinal disease.

The predictive capacity of the absorption test has the potential to identify animals at risk of future weight loss, where weight loss is a known prognostic factor for survival in a variety of diseases, e.g., CKD and CHF. Early diagnosis is crucial to a positive prognosis but also a tool with which treatment responses can be monitored allowing for adaptive and personalised therapeutic approaches. Validating lactulose/galactose absorption testing as a reliable tool for weight loss prediction would allow for its use in 48%-54% of canine CHF, 35% of oncology, and 61% of CKD patients where weight loss and cachexia are known to occur.

### Biosensor Systems

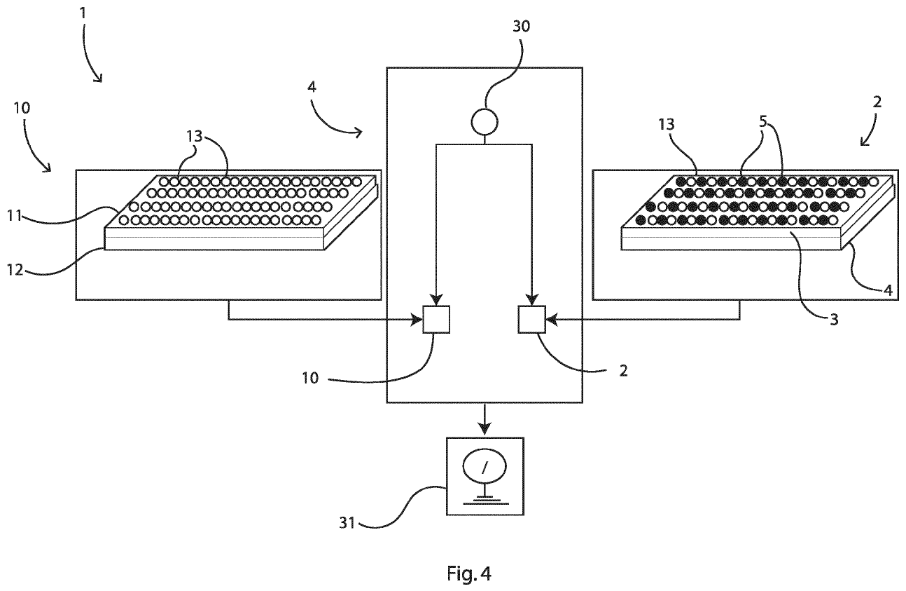

It is preferred that in measuring the concentration of both lactulose and galactose either a dual sensor system for the detection of lactulose and galactose (FIG. 4) is used or a single-sensor system is used.

### Step 1

Lactulose is converted into galactose using the enzyme β-galactosidase. The resultant galactose is subsequently converted into D-galacto-hexodialdose and hydrogen peroxide in the presence of oxygen or a redox mediator. The enzyme galactose oxidase is used to liberate hydrogen peroxide from galactose. Step 1 has the main steps of:
a) converting lactulose in the blood sample (for example plasma) into an entity including galactose, preferably using the enzyme β-galactosidase;
b) converting the galactose into D-galacto-hexodialdose and hydrogen peroxide in the presence of oxygen or a redox mediator;
c) detecting one or more of the following which are provided by step (b), and using it to determine a concentration of lactulose:
   quantity of hydrogen peroxide, and/or
   quantity of D-galacto-hexodialdose, and/or
   a change in the quantity of redox mediator, and/or
   a change in the quantity of oxygen.

In one case an electrochemical biosensor including a working electrode, a counter electrode and a reference electrode is provided, at least one enzyme is immobilized on the working electrode surface, the step (a) and (b) conversions are performed by contact with the immobilized enzyme or enzymes when the working electrode is exposed to the sample and a voltage or current is applied across the electrodes. The working electrode may include an electron mediator such as platinum (Pt) or ruthenium purple and the enzymes immobilised onto the working electrode surface.

FIG. 4 shows a dual biosensor system 1 having biosensors 2 and 10 with electron mediator working electrodes 3, 11 on a polymer substrate 4, 12 respectively. This diagram shows immobilised enzymes β-galactosidase 13 and Galactose Oxidase 5 on the electrode surface 3 and only β-galactosidase 13 on the electrode 11. The enzymes 5 and 13 are immobilized onto the working electrodes in some examples by cross linking, and/or silica sol-gel entrapment and/or co-disposition with polymer.

A well 30 provides via microfluidic channels sample to the sensors 2 and 10, and current is measured by an ammeter or voltammetry system 31. The sensors 2 and 10 are two biosensors applied to a ridged surface which is layered with a microfluidic cover. In one example the dual-sensor system 1 measures with the two immobilized enzymes including β-galactosidase 13 for the step (a) conversion and galactose oxidase 5 for the step (b) conversion.

In more detail, and referring again to FIG. 4, the biosensor 1 is a dual electrochemical biosensor cartridge with microfluidic cover and its overall dimensions are approximately 85.6 mm height and 53.98 mm width. The test cartridge is inserted into a reader prior to sample application.

Sample (plasma) is applied to the sample well 30. The sample travels by capillary action down both microfluidic channels to the electrochemical biosensors 2 and 10. Length and width of the microfluidic channel controls the volume of sample applied to the functionalised working electrode. This results in the working electrodes being parallel to each other. This can also be completed on a single electrode which contains two functionalised working electrodes like the electrodes 3 and 11 but configured in sequence.

In one case the hydrogen peroxide is oxidised on the working electrode surface in step (b). The hydrogen peroxide may be detected in step (c), and the detection is by detection of the current flow in the sensor.

In some cases, a redox mediator is used as an electron transporter between the enzyme(s) and the working electrode. If so, a change in the quantity of the redox mediator as a result of the conversion of the galactose into D-galacto-hexodialdose and hydrogen peroxide may be detected and processed to generate the output. The redox mediator may, for example, be selected from ferrocene and its derivatives, quinone-based compounds, ferro/ferricyanide, or various redox organic polymers or inorganic complexes or combinations thereof, and it may comprise osmium or ruthenium purple.

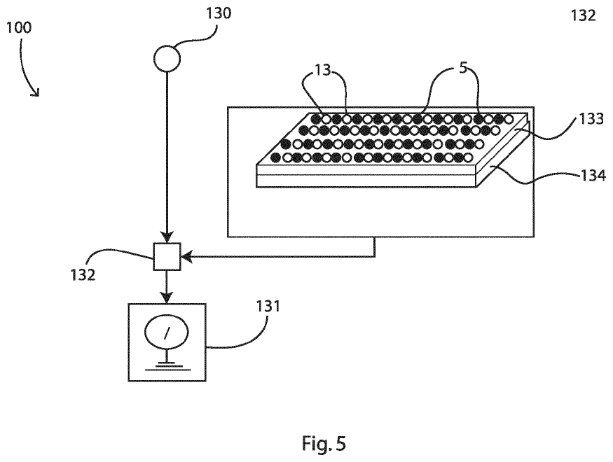

Data was generated using electrochemical biosensors which were prepared per Step 1. To evaluate the analytical performance of the prepared biosensor, an amperometric test was performed in phosphate buffer solution (10 mM, pH 7.4) with its corresponding substrate of 5 to 50 μM concentration. For experiments with plasma, frozen plasma was firstly thawed on ice, then spiked with 500 μM galactose or lactulose of a known concentration before use. The data shown in FIGS. 6 and 7 demonstrate the linear response of the sensor across the target range for both galactose and lactulose in plasma.

### Step 2

To measure galactose, the sample is converted into D-galacto-hexodialdose and hydrogen peroxide in the presence of oxygen or a redox mediator. The enzyme galactose oxidase 5 on the sensor 10 is used to liberate hydrogen peroxide from galactose.

The concentration of galactose measured in Step 1 (that is, galactose derived from lactulose) is subtracted from the concentration of galactose measured in Step 2, to avoid double-counting. Therefore, the sensor 2 in FIG. 4 gives a galactose and lactulose reading based on the hydrogen peroxide concentration determined enzymatically; and the sensor 10 in FIG. 4 gives a galactose only reading. Areas under the curve for lactulose and galactose can be calculated as described below:
Linear Trapezoidal Method in which linear interpolation between data points is used to calculate the AUC. In this instance, the average concentration (using linear methods) is applied to the entire time interval. The sum of the intervals results in the total exposure between the first and last time points. Dividing the total AUC by the total time elapsed results in an “average” concentration of drug in the body of over the total time interval.

$$AUC = \frac{1}{2} ( C 1 + C 2 ) ( t 2 - t 1 )$$

Logarithmic Trapezoidal Method: This is considered an accurate method of calculation when concentrations are decreasing as drug elimination is exponential. In this method, logarithmic interpolation between data points is used to calculate the AUC and assumes C1>C2.

$$AUC = ( ( C 1 - C 2 ) / ( ln ( C 1 ) - ln ( C 2 ) ) ( t 2 - t 1 )$$

Linear-Log Trapezoidal Method: Is a combination of the linear and logarithmic trapezoidal method and considered the most accurate as it incorporates the best approximation of drug absorption using the linear method and drug elimination using the logarithmic trapezoidal method.

In work completed to date, cumulative AUC values were determined for each sugar using the linear-log trapezoidal method across multiple sampling timepoints up to 1 hour post administration, and ratios of cumulative values at each timepoint used to derive information on gastrointestinal permeability. Ratios of AUCs at earlier timepoints provide information on gastric and upper small intestinal permeability, while later ratios may be more reflective of permeability in the GI tract generally. Adjusting later ratios using the early ratio values within individuals may allow further localisation within the GI tract and will be explored in future investigations.

### Single Biosensor System (100)

FIG. 5 shows a biosensor system 100 with only a single biosensor, having both enzymes described for the system 1. The same operating principles apply, but the steps are time separated. The single biosensor system 100 has a single biosensor 132 utilizing an electron mediator working electrode 133 on a polymer substrate 134. There are immobilised enzymes β-galactosidase 13 and Galactose Oxidase 5 on the working electrode 133. The enzymes are immobilized in some examples onto the working electrode 133, by cross linking, and/or silica sol-gel entrapment, and/or co-disposition with polymer.

A well 130 provides via microfluidic channels sample to the sensor 132, and current is measured by an ammeter system 131 or Voltammetry system. The biosensor 132 is applied to a ridged surface which is layered with or without a microfluidic cover. In one example the sensor system measures with two main steps as follows.

### Step 1

To measure endogenous galactose as well as galactose contained in the oral solution administered prior to sample draw, the sample (for example plasma) is converted into D-galacto-hexodialdose and hydrogen peroxide in the presence of oxygen or a redox mediator. The enzyme galactose oxidase 5 is used to liberate hydrogen peroxide from galactose. This is measured on the sensor working electrode 133 immediately after sample application.

### Step 2

Lactulose is converted into galactose using the enzyme β-galactosidase. The resultant galactose is subsequently converted into D-galacto-hexodialdose and hydrogen peroxide in the presence of oxygen or a redox mediator. The enzyme galactose oxidase is used to liberate hydrogen peroxide from galactose. Step 2 has the main steps (a), (b), and (c) set out above.

In some cases, a redox mediator is used as an electron transporter between the enzyme(s) and the working electrode. If so, a change in the quantity of the redox mediator as a result of the conversion of the galactose into D-galacto-hexodialdose and hydrogen peroxide may be detected and processed to generate the output. The redox mediator may, for example, be selected from ferrocene and its derivatives, quinone-based compounds, ferro/ferricyanide, or various redox organic polymers or inorganic complexes or combinations thereof, and it may comprise osmium or ruthenium purple.

To measure galactose, the sample (for example plasma) is converted into D-galacto-hexodialdose and hydrogen peroxide in the presence of oxygen or a redox mediator. The enzyme galactose oxidase 5 is used to liberate hydrogen peroxide from galactose. This is measured on the sensor 132. The concentration of galactose measured from the initial reading measured in step 1 (that is, endogenous galactose as well as galactose contained in the oral solution administered prior to sample draw) is subtracted from the concentration of galactose measured in Step 2, to avoid double-counting. Therefore, the initial reading gives a galactose only reading and the second reading gives a galactose and lactulose reading based on the hydrogen peroxide concentration determined enzymatically. Areas under the curve for lactulose and galactose can be calculated as set out above.

Alternative methods of analysis and detection that may be used to measure individual galactose and lactulose concentrations in blood are described in Examples 5 and 6.

### Example 5—Detection of Both Galactose and Lactulose Using High Performance Liquid Chromatography (HPLC) Followed by Mass Spectroscopy (MS)

Samples are analysed using an Agilent 1060 System, with quaternary solvent delivery system, automated injection, and mass spectrometer detection. A Phemonex Luna® NH2 analytical column 5-micron, 150×2 mm, or an equivalent column, can be used for separation. The mobile phase can consist of acetonitrile and water, with a flow rate of 0.3 mL/min. Detection is monitored using m/z (mass to charge ratio) in negative ionization mode of 341 with additional detection of daughter fragment ions at 160.7 and 100.9 m/z. Integration of chromatograms can be performed with Agilent OpenLab® software or an equivalent data capture system.

The concentrations of galactose and lactulose are processed to provide a concentration ratio. This processing may use a Linear Trapezoidal Method, a Logarithmic Trapezoidal Method, or Linear-Log Trapezoidal Method as described above.

### Example 6—Impedimetric Detection of Both Galactose and Lactulose

Impedimetric detection of both galactose and lactulose is based on 1) a lactulose-binding lectin and 2) a galactose-binding lectin. The quantitative detection of lactulose and galactose is based on a competition reaction between the target sugar in the blood sample (target analyte) and large molecule complexes, in this case sugar derivatives, bound to the specific binding lectin. Sugar derivatives such as β-galactosidase containing a functional group at the hydroxy end of the lactulose sugar can be utilised for lactulose quantification. Sugar derivatives such as β-galactosidase containing a functional group at the terminal end can be utilised for galactose quantification. The amount of sugar derivatives bound to the specific-binding lectin on an electrode (eg. Gold electrode) surface is inversely proportional to the amount of the target sugar (target analyte) in the sample solution. These derivatives are much larger than the targets and so provide an easily detectable impedimetric response. This is despite the fact that the sample is a complex matrix.

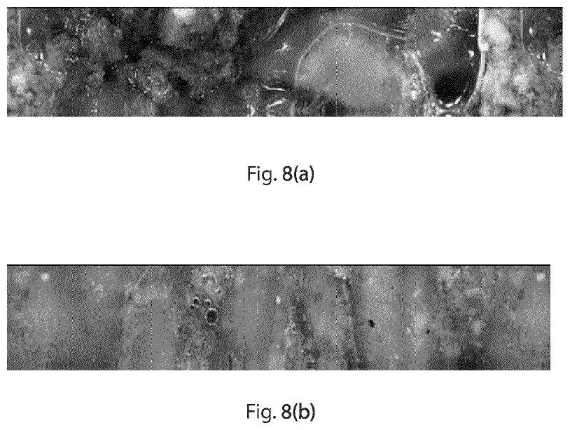

FIG. 9 shows a dual electrochemical sensor system 51 having three-electrodes (working, reference, and counter electrodes) electrochemical sensors 52 and 60 with target sugar specific binding lectins 53, 61 on a polymer substrate 54, 62 respectively. This diagram shows immobilised lactulose binding lectin 63 on an electrode surface 61 and galactose binding lectin 55 on the electrode surface 53. The target sugar specific lectins 55 and 63 are immobilized onto the working electrodes in some examples by cross linking, and/or silica sol-gel entrapment and/or co-disposition with polymer.

A well 80 provides sample to the sensors 52 and 60 via microfluidic channels, and current is measured by an ammeter or voltammetry system 81. The sensors 52 and 60 are two electrochemical sensors applied to a ridged surface which is layered with a microfluidic cover. In one example the dual-sensor system 51 measures with the two immobilized target sugar specific lectins.

The more of the derivatives which bind to the lectin probes (binding site) the less target analyte is present.

A galactose-binding lectin is described by Jong and Lee in Journal of Electroanalytical Chemistry 903 (2021) 115846.

A lactulose-binding lectin is described by Kawagishi et al Biosci. Biotechnol. Biochem. 2001 November; 65 (11): 2437-42.

### Example 7: Competitive ELISA (Enzyme Linked Immunosorbent Assay)

This assay uses the same complexes as are used in the impedimetric approach above, but in this case a monoclonal antibody is attached to the complexes for optical detection.

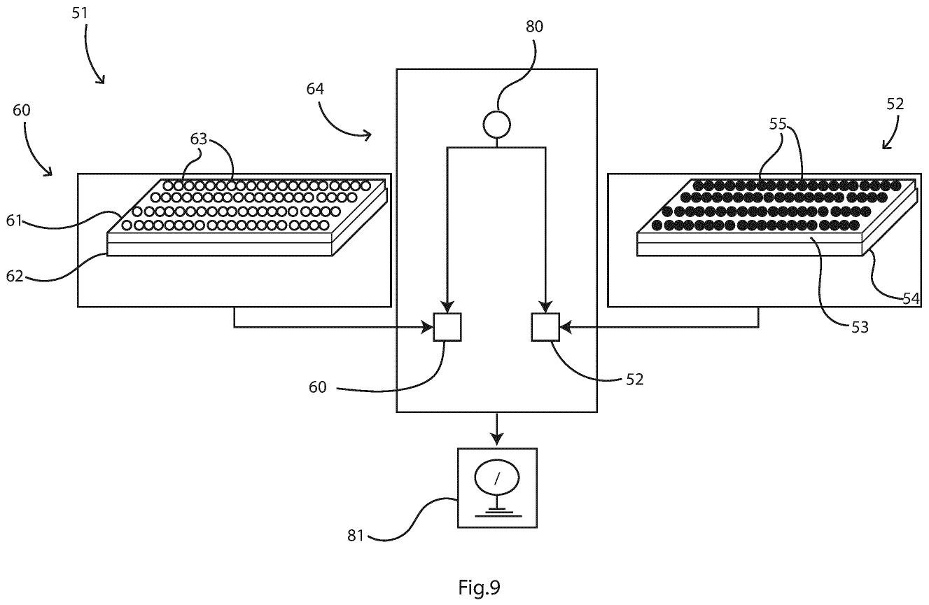

Galactose specific binding lectin and lactose specific binding lectins are utilised to capture the target sugar of interest from the blood sample. The detection and quantification process for galactose is illustrated in FIGS. 10(a) to 10(d). A micro well 200 has a substrate 201 which is coated with galactose specific binding lectin 202. When the lectin 202 has had sufficient time to bind to the micro well substrate 201 any unbound lectins are removed from the well via rinsing. A sample (plasma or blood) is then added to the micro well 200. Any galactose in the sample binds to the galactose specific binding lectin 202 in the micro well. Galactose binding to the lectin 202 is illustrated by reference 203 (FIG. 10(b)). Following capture of the target analyte the well is rinsed leaving the lectin and target analyte in the well. A target sugar/protein complex 204 in this case is a galactose molecule containing a protein at the terminal end. The sugar protein complex 204 binds to any residual galactose binding lectin 202 in the micro well as illustrated in FIG. 10(c). Surplus galactose protein complex 204 is washed from the micro well prior to adding a monoclonal antibody 205 to the micro well. The monoclonal antibody 205 specifically binds to the protein portion of the sugar protein complex 204 (FIG. 10(d)), and unbound antibody is removed from the well by rinsing. Each monoclonal antibody 205 will be conjugated to a molecule that will facilitate quantification of the bound monoclonal antibody 205 in the micro well. One example of this molecule is an enzyme conjugated antibody. In this case, when the enzyme acts on its substrate a colour change occurs which is proportional to the concentration of the antibody in the micro well. In this instance the final step of the galactose quantification is to add the enzyme substrate to the micro well and measure the colour intensity using a UV-Visible spectrophotometer. The concentration of galactose in the sample will be inversely proportional to the signal as where no galactose was present in the blood sample all of the available galactose specific binding lectins will have a bound galactose protein complex 204. As per the example above where 100% of the lectins are bound to the sugar protein complex it will result in maximal colour change in the final step FIG. 10(d) which would be equivalent to the control micro well. The control will follow the process in FIG. 10 with the exception of FIG. 10(b) as no sample will be added to the micro well. Where a sample contains galactose it will bind with the galactose specific binding lectin resulting in fewer binding locations for the galactose protein complex as such the resulting colour change from the enzyme substrate interaction will be reduced.

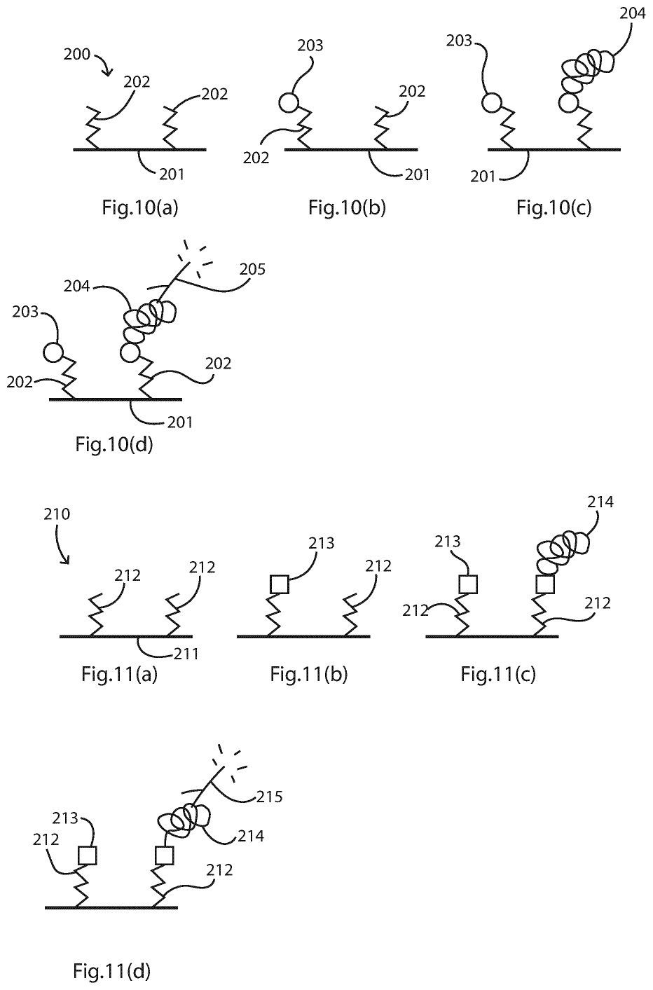

Referring to FIGS. 11(a) to 11(d), quantification of lactulose in the sample is determined by using a similar process to quantification of galactose. A micro well 210 has a substrate 211 which is coated with lactulose specific binding lectin 212. When the lectin 212 has had sufficient time to bind to the micro well substrate 211 any unbound lectins are removed from the well via rinsing. A sample (plasma or blood) is then added to the micro well 210. Any lactulose in the sample binds to the lactulose specific binding lectin 212 in the micro well. Lactulose binding to the lectin 212 is illustrated by reference 213 (FIG. 11(b)). Following capture of the target analyte the well is rinsed leaving the lectin and target analyte in the well. A target sugar protein complex 214 in this case is a lactulose molecule containing a protein at the terminal end. The sugar protein complex 214 binds to any residual lactulose binding lectin 212 in the micro well as illustrated in FIG. 11(c). Surplus lactulose protein complex 214 is washed from the micro well prior to adding a monoclonal antibody 215 to the micro well. The monoclonal antibody 215 specifically binds to the protein portion of the sugar protein complex 214 (FIG. 11(d)), and unbound antibody is removed from the well by rinsing. Each monoclonal antibody 215 will be conjugated to a molecule that will facilitate quantification of the bound monoclonal antibody 215 in the micro well. One example of this molecule is an enzyme conjugated antibody whereby when the enzyme acts on its substrate a colour change occurs which is proportional to the concentration of the antibody. In this instance the final step of the lactulose quantification is to add the enzyme substrate to the micro well and measure the colour intensity via UV-Visible spectrophotometer. The concentration of lactulose in the sample will be inversely proportional to the signal as where no lactulose was present in the blood sample all of the available lactulose specific binding lectins will have a bound lactulose protein complex 214. As per the example above where 100% of the lectins are bound to the sugar protein complex it will result in maximal colour change in the final step FIG. 11(d) which would be equivalent to the control micro well. The control will follow the process in FIG. 11 with the exception of FIG. 11(b) as no sample will be added to the micro well.

Where a sample contains lactulose, it will bind with the lactulose specific binding lectin resulting in fewer binding locations for the lactulose protein complex as such the resulting colour change from the enzyme substrate interaction will be reduced.

The concentrations of galactose and lactulose are processed to provide a concentration ratio. This processing may use a Linear Trapezoidal Method, a Logarithmic Trapezoidal Method, or Linear-Log Trapezoidal Method as described above.

On obtaining such measurements indicating gastric permeability in a canine patient, for example, gastric ulceration, a veterinarian may prescribe therapy in the form of a proton pump inhibitor. Omeprazole extended-release oral capsules are currently a preferred treatment option and are dosed at 0.5 to 1.0 mg/kg bodyweight, twice daily, for 21 days. Repeat testing as described above may be conducted on completion of therapy to ensure resolution of gastric ulceration. Human and equine patients may be similarly treated, but at dose rates of 0.5 and 4 mg/kg respectively, once or twice daily as deemed necessary by the attending clinician. These and other treatments that may be used after detection of gastric ulceration as described herein are outlined in detail below.

Point-of-care testing represents an easy to use, economical alternative to gastroscopy for the diagnosis of gastric ulcers and in monitoring the quality of ulcer healing. Importantly, existing methods of determining both presence and resolution of ulceration rely predominantly on visual assessment of the intactness of the gastrointestinal mucosa by endoscopy in patients. This approach has led to the assumption that the mucosa of grossly “healed” gastric and/or duodenal ulcers returns to normal, either spontaneously or following treatment. However, the re-epithelialized mucosa of grossly “healed” experimental gastric ulcer has been found to have prominent histologic and ultrastructural abnormalities, including reduced height, marked dilation of gastric glands, poor differentiation and/or degenerative changes in glandular cells, increased connective tissue, and disorganized microvascular network. These residual abnormalities interfere with mucosal defence and may be the basis of ulcer recurrence. Recent studies have shown that prominent histologic, structural, and gene expression abnormalities in macroscopically healed gastric epithelium. These studies also showed that the regenerated epithelium after ulcer healing remains abnormal for months after healing and suggested that the sites of ulcer scars are where ulcers recur.

Functional and structural assessment of the quality of ulcer healing is currently only possible in experimental settings or by obtaining tissue samples by biopsy. However, biopsy necessitates again disrupting the mucosal barrier making it an undesirable intervention.

The invention provides a non-invasive method to assess gastrointestinal permeability not only for diagnosis of gastric ulcers but also for ongoing monitoring of the health of gastrointestinal mucosa and submucosa at the site of previous injury, allowing healthcare professionals to provide individualised, risk-based care to their human or veterinary patients

### Gastric Ulceration

The monogastric stomach is an active reservoir that stores, triturates, and slowly dispenses partially digested food into the intestine for further digestion and absorption, and also controls appetite and satiety. Its main secretory function is secretion of gastric acid, which initiates peptic hydrolysis of dietary proteins, liberates vitamin B12 from dietary protein, facilitates duodenal absorption of inorganic iron and calcium, stimulates pancreatic HCO₃— secretion via secretin release, suppresses antral gastrin release, and modulates the intestinal microbiome by killing microorganisms and preventing bacterial overgrowth.

Dogs have lower basal but higher peak gastric acid secretion compared to humans. However, fasting gastric pH is comparable in dogs and humans. In one study the intragastric pH of healthy control dogs remained <2.0 over 85% of the time, with a mean percentage time (MPT) intragastric pH>4.0 of only 4.7% (Tolbert K, Bissett S, King A, et al. Efficacy of oral famotidine and 2 omeprazole formulations for the control of intragastric pH in dogs. J Vet Intern Med. 2011; 25:47-54). In another study, the median gastric pH was 1.1 and the median percentage of the investigation time that the gastric pH fluctuated between 0.5 and 2.5 was 90.32% (range, 78%-97.4%) (Kook P H, Kempf J, Ruetten M, Reusch C E. Wireless ambulatory esophageal pH monitoring in dogs with clinical signs interpreted as gastroesophageal reflux. J Vet Intern Med. 2014; 28:1716-1723).

Gastric pH in humans increases with feeding because of the buffering effect of food, but dogs differ because the buffering effect of food is not consistently observed and is much smaller in effect, if present at all. This observation may be caused by higher peak acid output in fed dogs. Another explanation may be differences in methodology, because the pH capsule methodology used in newer studies, unlike digital probes, allows direct adherence to the gastric mucosa and provides direct measurement of intragastric pH.

The horse stomach continuously secretes variable amounts of hydrochloric acid throughout the day and night and secretion of acid occurs without the presence of feed material. Foals secrete gastric acid as early as 2 days of age and acidity of the gastric fluid is high. High acid in the stomach may predispose foals to gastric ulceration. The adult equine stomach secretes approximately 1.5 litres of gastric juice hourly and acid output ranges from 4 to 60 mmoles hydrochloric acid per hour. The pH of gastric contents ranges from 1.5 to 7.0, depending on region measured. A near neutral pH can be found in the dorsal portion of the esophageal region near the lower oesophageal sphincter, whereas more acidic pHs can be found in the glandular region near the pylorus (1.5-4.0). Gastric emptying of a liquid meal occurs within 30 minutes, whereas complete gastric emptying of a roughage hay meal occurs in 24 hours.

### Gastric Mucosal Barrier

The gastric mucosal barrier is a complex defence mechanism, protecting the normal mucosa from the harsh chemical environment of the gastric luminal contents. Gastric luminal peptides and gastric distention provide strong stimulation for gastric acid production. In response to stimulation, parietal cell H+/K+-ATPase and KCl transporters become incorporated into the parietal cell canalicular membrane. Hydrogen ions are released into the gastric lumen from parietal cells upon stimulation in exchange for potassium, resulting in a very acidic environment.

The gastric mucosal barrier protects the gastric epithelium from the highly acidic luminal contents. Tight junctions seal the cellular layers of the gastric mucosa, ensuring that the luminal contents do not leak into or around these cells. A thick, bicarbonate-rich mucous layer covers the epithelial surface. The small amount of gastric acid that diffuses into epithelial cells is quickly cleared by the high blood flow to this area. This high blood flow also supports cellular metabolism and rapid renewal of injured cells. Local production of prostaglandins E2 and I2 help maintain the GI mucosal blood flow and integrity, increase mucous and bicarbonate secretion, decrease acid secretion, and stimulate epithelial cell turnover.

In the normal GI tract, the potential disruptive properties of the luminal contents are balanced by the defence mechanisms of the GI mucosal barrier. However, many drugs and diseases have the potential to upset the balance between the harsh luminal contents and the GI protective barrier. GI ulceration primarily targets the stomach and/or duodenum.

A defect in the normal GI mucosal barrier leads to a self-perpetuating cycle of further mucosal damage. Injury to this barrier allows hydrochloric acid, bile acids, and proteolytic enzymes to degrade the epithelial cells, disrupt lipid membranes, and induce inflammation and apoptosis. Back diffusion of luminal contents through the tight junctions leads to inflammation and haemorrhage of the GI cells, with further acid secretion mediated by inflammatory cells and their products. Mast cell degranulation occurs, causing histamine release that perpetuates further gastric acid secretion. The inflammatory environment also causes decreased blood flow, resulting in ischemia, decreased ability for cellular repair, and reduced secretion of mucus and cytoprotective prostaglandins. Mucosal ulceration can result, exposing the submucosa or deeper layers of the GI tissue to the harsh chemical luminal contents.

### Aetiology of Gastric Ulceration in Different Species

### Horses

Ulcers in the non-glandular squamous mucosa are associated with repeated direct insult from ultra-low pH fluid normally found in the glandular region of the stomach in horses. Pressure increases inside the abdomen (associated with exercise), collapsing the stomach and forcing the acid gastric contents upward. The more fluid (and highly acidic) contents of the lower stomach come in contact with the non-glandular squamous mucosa, causing inflammation and, potentially, erosions to varying degrees.

The causes of ulcers in the glandular mucosa of the stomach are less well defined. Use of nonselective NSAIDs are known to reduce blood flow to the GI tract, causing decreased production of the muco-bicarbonate matrix by the gastric glandular mucosa and resulting in ulceration. This is not a consistent finding, however. Additionally, attempts have been made to isolate and/or correlate evidence of Helicobacter organisms from the stomach of horses with and without gastritis and ulcers. Results of these studies have been equivocal or negative, and the role of this organism in glandular equine gastric ulcers has not been determined.

### Humans

Helicobacter pylori and NSAIDs disrupt normal mucosal defence and repair, making the mucosa more susceptible to acid. H. pylori infection is present in 50 to 70% of patients with duodenal ulcers and in 30 to 50% of patients with gastric ulcers. If H. pylori is eradicated, only 10% of patients have recurrence of peptic ulcer disease, compared with 70% recurrence in patients treated with acid suppression alone. NSAIDs now account for >50% of peptic ulcers.

Cigarette smoking is a risk factor for the development of ulcers and their complications. Also, smoking impairs ulcer healing and increases the incidence of recurrence. Risk correlates with the number of cigarettes smoked per day. Although alcohol is a strong promoter of acid secretion, no definitive data link moderate amounts of alcohol to the development or delayed healing of ulcers. Very few patients have hypersecretion of gastrin caused by a gastrinoma (Zollinger-Ellison syndrome).

### Dogs

In dogs, NSAID administration, neoplasia, and hepatic disease are the most common reported causes of gastric ulceration. NSAIDs can cause direct topical damage to the GI mucosa, and inhibition of cyclooxygenase (COX)-1 decreases production of protective prostaglandins. The use of COX-2-specific NSAIDs is thought to decrease GI ulceration, but ulceration and perforation can still occur with use of these medications.

Primary GI neoplasia such as lymphoma, adenocarcinoma, leiomyoma, and leiomyosarcoma can result in ulceration due to local effects of the tumour. Additionally, paraneoplastic syndromes secondary to mast cell tumours and gastrinomas (Zollinger-Ellison syndrome) have been associated with increased gastric hydrochloric acid production and subsequent ulceration in dogs.

Various hepatic diseases (e.g., acute hepatic injury, intrahepatic portosystemic shunt) are associated with gastroduodenal ulceration, but the mechanism of disease is not known. Possible causes include increased gastric acid secretion and alterations in mucosal blood flow, potentially leading to ulcer formation.

Other causes of ulceration in dogs include major trauma, spinal disease, renal disease, hypoadrenocorticism, GI inflammation such as inflammatory bowel disease or presence of a traumatic foreign body, systemic inflammation such as pancreatitis and sepsis, and extreme exercise such as sled dog racing. Combining NSAID and corticosteroid therapy will increase risk of GI ulceration and is contraindicated.

### Treatment of Gastric Ulceration

Treatment of gastric ulceration follows a common approach across monogastric species.

### Elimination of Predisposing Lifestyle, Dietary and Stress Factors

In humans, lifestyle changes such as cessation of smoking and reducing or managing stress may aid in resolution of gastric ulceration and prevention of recurrence. In horses, a break from training and alterations to the diet to increase the amount of roughage consumed may similarly aid recovery.

### Elimination of Causative Medications

NSAIDs are a common cause of gastric ulceration in all treated monogastric species. A recent study has shown that 75% of dogs receiving chronic NSAID therapy have overt or silent gastric ulceration, while approximately 12% of human patients likewise develop gastric lesions within the first 12 weeks of NSAID therapy. In asymptomatic cases, NSAIDs may justifiably be continued in the interest of managing an underlying inflammatory or painful disorder, but administration should cease in human, equine, canine and other patients presenting with symptoms of ulceration. Dysbiosis that occurs as a result of therapeutic gastric pH suppression may potentiate the damaging effects of NSAIDs on gastric and intestinal mucosa, and so co-administration of gastric acid suppressants and NSAIDs should be avoided. There is conflicting evidence as to the ulcerogenic potential of corticosteroids when used as monotherapy in all species, but NSAID-corticosteroid combination therapy has potent effects in weakening the gastric mucosal barrier and ulcer development.

### Surgery

Surgical excision of deep and/or perforated gastric ulcers is sometimes performed in all species, with primary repair of the gastric wall by apposing healthy tissue beyond the ulcer margins. However, due to high perioperative morbidity and mortality the procedure is carried out only by necessity and is best avoided through early intervention and aggressive medical therapy. When gastric ulceration occurs due to a neoplastic process such as gastrinoma, surgical excision of the tumour may be curative.

### Antisecretory Medications

Reduction of acid secretion and the resultant increase of gastric pH are fundamental to successful treatment of gastric ulceration. In humans, healing of gastric ulcers is highly correlated with the degree of gastric acid suppression. For people with ulcers, optimal treatment involves maintaining an intragastric pH≥3 for 18 to 20 hours a day (i.e., approx. 75% of the day) and little benefit is associated with more extensive gastric acid suppression. Intragastric pH>6 is necessary to achieve haemostasis with acute gastrointestinal tract bleeding, as pH values >6 allow adequate platelet aggregation and prevent dissolution of blood clots.

The optimal degree of gastric acid suppression necessary for treating acid-related diseases in other monogastric species has not been established, and critical intragastric pH thresholds in dogs and horses may differ from those established for humans because of differences in gastric physiology. Regardless, it seems likely that the degree and duration of gastric acid suppression will correlate with ulcer healing in all monogastric species. Assessment of ulcer healing may be conducted using gastroscopy; however, this simply establishes re-epithelialisation and as discussed above cannot assess the quality of ulcer healing. Ulcer recurrence has been reported in cases in which the quality of healing is poor, and early identification of such cases offers the opportunity for early medical and lifestyle interventions to optimise outcomes for patients. The invention provides a non-invasive method for monitoring of gastrointestinal permeability in patients with a history of ulceration within the previous 12 months and has the potential to reduce patient morbidity and mortality.

### Antacids

Antacids are the oldest of the gastrointestinal (GI) protectants and comprise a group of inorganic, relatively insoluble salts of aluminium hydroxide (Al(OH)₃), calcium carbonate (CaCO₃), and magnesium hydroxide (Mg(OH)₂) that lack systemic effects. Antacids may be beneficial by decreasing pepsin activity, binding to bile acids in the stomach, and stimulating local prostaglandin synthesis. Antacids have historically been used in humans and dogs but are ineffective in the treatment of gastric ulceration. Constipation is the most common adverse effect in both species.

### Histamine Type-2 Receptor Antagonists

Histamine type-2 receptor antagonists (H2RAs; e.g., cimetidine, ranitidine, and famotidine) inhibit acid secretion by competitively blocking H-2 receptors on the parietal cell, thus decreasing gastric acid secretion. Continuous H2RA administration results in pharmacological tolerance within days, which can be demonstrated through gastric pH monitoring. Tolerance occurs even more rapidly (12-72 hours) in human subjects when famotidine is administered intravenously. Because of this tolerance, abrupt discontinuation of H2RAs causes rebound acid hypersecretion in humans as a result of the trophic properties of gastrin on enterochromaffin like cells. This phenomenon has not yet been documented in dogs or horses, although it appears reasonable to assume its occurrence. The H2RAs have been shown to increase gastric pH in all monogastric species evaluated to date, though appear to be less effective than proton pump inhibitors (PPIs).

### Proton Pump Inhibitors

The PPIs (e.g. omeprazole, pantoprazole, esomeprazole, and lansoprazole) are substituted benzimidazole drugs that target the final common pathway of acid production. The PPIs are significantly more effective than H2RAs in increasing gastric pH and preventing and healing gastric ulceration. The PPIs irreversibly inactivate the acid secretory pathway, resulting in a prolonged effect after administration. Maximal inhibitory effect is achieved within approximately 2-4 days of PPI administration. With repeated dosing, omeprazole may reduce its own metabolism, increasing its effectiveness through inhibition of cytochrome P450 enzymes. While several PPIs (e.g. omeprazole, esomeprazole, lansoprazole, pantoprazole) have been approved for use in human gastric ulcer patients, only omeprazole has received regulatory approval for use in horses, and no PPI has been approved for use in dogs or other monogastrics to date. It has been consistently shown that PPIs are superior to H2RAs for increasing intragastric pH and facilitating gastric ulcer healing. For all monogastric species, it has been suggested that PPIs should be gradually tapered after administration for ≥4 weeks to avoid rebound gastric acid hypersecretion.

### Sucralfate

Sucralfate (Carafate) is a complex salt of sucrose octasulphate and aluminium hydroxide that may be safely used in the treatment of gastric ulceration in humans, dogs, and horses. Its mechanism of action in acid-peptic disease is multifactorial. Sucralfate forms stable complexes with protein in damaged mucosa where there is a high concentration of protein, either from fibrinogen, albumin, or globulins from the exudate of an ulcer or from damaged cells. In an acidic environment, sucralfate becomes viscous and partially dissociates into sucrose sulphate and aluminium hydroxide. The sucrose sulphate moiety is an anion and binds electrostatically with the positively charged proteins in the damaged mucosa. Sucralfate interferes with the action of pepsin either by preventing pepsin digestion of protein substrates, by binding to pepsin, or by providing a barrier to prevent diffusion of pepsin. In addition, the protection afforded by sucralfate against oesophageal acid injury is mediated by intraluminal pH buffering via aluminium hydroxide and protection against H+ entry and injury via sucrose octasulphate. Sucralfate also may provide a barrier for bile salts. Sucralfate is known to stimulate prostaglandin production in the gastric epithelium. This may be a potential secondary effect of sucralfate in the oesophagus, although the importance and effectiveness of sucralfate as an agent for the treatment of erosive esophagitis is not as established as it has been for H2RAs or PPIs. In foals, sucralfate had a protective effect on gastric ulcers associated with intravenous administration of high-dose NSAIDs.

### Combination Therapy for Helicobacter

In humans, H. pylori is implicated in the majority of cases of gastric ulceration. While non-H. pylori Helicobacter infection has been reported in other monogastric species, the significance of these infections and the clinical benefit of eliminating them remains questionable. Anti-H. pylori therapy in humans currently consists of multiple drugs, either simultaneously or sequentially, and PPIs are almost always an integral component of treatment.

While choosing a treatment regimen for H. pylori, human patients should be asked about previous antibiotic exposure and this information should be incorporated into the decision-making process. For first-line treatment, clarithromycin triple therapy should be confined to patients with no previous history of macrolide exposure who reside in areas where clarithromycin resistance amongst H. pylori isolates is known to be low. Most patients are better served by first-line treatment with a PPI, bismuth subcitrate, tetracycline and metronidazole (bismuth quadruple therapy) or a PPI, clarithromycin, amoxicillin, and metronidazole for 10 to 14 days. When first-line therapy fails, a salvage regimen should avoid antibiotics that were previously used. If a patient received a first-line treatment containing clarithromycin, bismuth quadruple therapy or salvage regimens that include levofloxacin are the preferred treatment options. If a patient received first-line bismuth quadruple therapy, clarithromycin or levofloxacin-containing salvage regimens are the preferred treatment options. The 3-year recurrence rate for gastric ulcers in humans is <10% when H. pylori is successfully eradicated but is >50% when it is not. Thus, a patient with recurrent disease should be tested for H. pylori and treated again if the tests are positive.

The references mentioned in this specification are herein incorporated by reference in their entirety.

The invention is not limited to the embodiments hereinbefore described, which may be varied in construction and detail.

1. A method for detecting gastrointestinal permeability in a subject comprising the steps of:
providing a sample of blood from a subject to which a solution comprising lactulose and galactose has previously been administered; and
in vitro determining a ratio of the concentration of lactulose to the concentration of galactose in the blood sample as an indicator of gastrointestinal permeability.

2. The method of claim 1, further comprising the step of providing a plurality of blood samples over a period of time after administering the solution.

3. The method of claim 2, wherein the period of time is at least 30 minutes.

4. (canceled)

5. The method of claim 1, further comprising converting the blood sample into plasma and determining the concentration of lactulose and galactose in the blood plasma.

6. The method of claim 5, wherein a determined ratio of the concentration of lactulose to the concentration of galactose of more than 1.1 in the blood plasma sample within 60 minutes after administration of the solution is indicative of increased gastrointestinal permeability.

7. The method of claim 1, wherein the lactulose is administered in the solution at an amount of 0.2 to 1.0 gram per kg bodyweight of the subject.

8. (canceled)

9. The method of claim 1, wherein the galactose is administered in the solution at an amount of from 0.01 to 1.0 gram per kg bodyweight of the subject.

10. (canceled)

11. The method of claim 1, wherein the solution comprises from 10% to 30% w/v of lactulose.

12. (canceled)

13. The method of claim 1, wherein the solution comprises from 1% to 5% w/v of galactose.

14-17. (canceled)

18. The method of claim 1, further comprising converting lactulose in the blood sample into an entity including galactose and processing the galactose to generate an output which is representative of the concentration of lactulose in the blood sample.

19. The method of claim 1, further comprising processing galactose in the blood sample to generate an output which is representative of the concentration of galactose in the blood sample.

20. The method of claim 19, wherein the processing comprises converting the galactose into D-galacto-hexodialdose and hydrogen peroxide in the presence of oxygen or a redox mediator and detecting one or more of the following which are provided:
quantity of hydrogen peroxide, and/or
quantity of D-galacto-hexodialdose, and/or
a change in the quantity of redox mediator, and/or
a change in the quantity of oxygen; and
processing the detected quantity or change in quantity to generate an output which is representative of the concentration of lactulose in the blood sample.

21-26. (canceled)

27. The method of claim 1, wherein an electrochemical biosensor system with a working electrode and a reference electrode is provided, at least one enzyme is immobilized on the working electrode surface and the conversion(s) are performed by contact with the immobilized enzyme or enzymes when the working electrode is exposed to the sample and a voltage is applied across the electrodes.

28-35. (canceled)

36. The method of claim 1, further comprising detection of lactulose based on a lectin that binds lactulose and detection of galactose based on a lectin that binds galactose.

37. (canceled)

38. The method of claim 1, further comprising processing the blood sample and testing by High Performance Liquid Chromatography (HPLC) followed by Mass Spectroscopy (MS) detection to generate an output which is representative of the concentration of lactulose and galactose in the blood sample.

39-64. (canceled)

65. An apparatus for determining a ratio of the concentration of lactulose to the concentration of galactose in a blood sample, the apparatus comprising:
a first converter for:
converting lactulose in the blood sample into an entity including galactose; and
converting the galactose into D-galacto-hexodialdose and hydrogen peroxide in the presence of oxygen or a redox mediator;
a first detector for detecting:
one or more of the following which are provided by the converter:
quantity of hydrogen peroxide, and/or
quantity of D-galacto-hexodialdose, and/or
a change in the quantity of redox mediator, and/or
a change in the quantity of oxygen;
a second converter for:
converting galactose in the blood sample into D-galacto-hexodialdose and hydrogen peroxide in the presence of oxygen or a redox mediator;
a second detector for detecting:
one or more of the following which are provided by the converter:
quantity of hydrogen peroxide, and/or
quantity of D-galacto-hexodialdose, and/or
a change in the quantity of redox mediator, and/or
a change in the quantity of oxygen;
and
a processor for:
processing the detected quantity or change in quantity provided by the detectors to generate an output which is representative of the ratio of the concentration of lactulose in the blood sample to the concentration of galactose in the blood sample.

66. The apparatus of claim 65, wherein each converter comprises a substrate for an enzyme to perform conversion of lactulose in the blood sample into galactose.

67. The apparatus of claim 66, wherein the enzyme for converting lactulose in the blood sample into galactose is β-galactosidase.

68. The apparatus of claim 65, wherein each converter comprises a substrate for an enzyme for conversion of the galactose into D-galacto-hexodialdose and hydrogen peroxide.

69. The apparatus of claim 68, wherein the enzyme for converting the galactose into D-galacto-hexodialdose and hydrogen peroxide is galactose oxidase.

70-88. (canceled)
